(19) United States
(12) Patent Application Publication
(10) Pub. No.: US 2003/0130693 A1
(43) Pub. Date: Jul. 10, 2003

(54) Laparoscopic/thorascopic insertion caps

(76) Inventors: John M. Levin, Narberth, PA (US); Matthew Wallash Levin, Narberth, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/371,015

(22) Filed: Feb. 20, 2003

Related U.S. Application Data
(63) Continuation-in-part of application No. 09/571,913, filed on May 16, 2000, now abandoned.
(60) Provisional application No. 60/134,638, filed on May 18, 1999.

(51) Int. Cl. A61B 17/28

(52) U.S. Cl. 606/205

(57) ABSTRACT

A laparoscopic/thorascopic insertion device is inserted into a body cavity and provides a passageway for inserting a laparoscopic/thorascopic clamp, dissector, or other surgical device without the use of a separate trocar. The insertion device includes a cylindrical shaft having a central passage extending inwardly from a proximal end of the insertion device for frictionally receiving a surgical device. A distal end of the insertion device has a conical shape terminating at a blunt tip adapted to safely displace muscle fibers, blood vessels, and delicate organs and body parts. In a preferred embodiment, the distal end is provided with a gripping area or region that can be engaged by a removal tool that initially is directed through a trocar inserted into the body cavity at an adjacent area of the body cavity.

### FIELD OF THE INTENTION

This invention relates generally to surgical instruments, and more specifically, to laparoscopic and thorascopic instruments that provide access to a body part.

### BACKGROUND OF THE INVENTION

In laparoscopic and thorascopic procedures, it is quite common to provide a number of passages through the abdomen and thorax to receive a camera, operating instruments, and clamps or other manipulating devices for engaging a body part to be acted upon by the operating instruments. Generally a trocar is inserted into the passages to maintain a constantly open passage in the patient for the repetitive insertion and removal of various devices and instruments. It is a desirable benefit in laparoscopic/thorascopic surgical procedures to minimize the number of trocars to be used, since they are quite expensive. In fact, especially with respect to graspers and other supporting devices, which are not repetitively inserted and removed during a surgical procedure, a trocar for repetitive use of the supportive devices generally is not required.

References throughout this application to “laparoscopic,” in referring either to a surgical procedure or a surgical device, or instrument, is intended to include within its scope thorascopic procedures, devices or instruments, unless the context of the reference to laparoscopic clearly indicates otherwise.

### SUMMARY OF THE INVENTION

Accordingly, it is an object of the invention to substantially obviate at least one of the problems associated with the related art.

It is also an object of the invention to provide a laparoscopic/thorascopic support device.

It is also an object of the invention to provide simple and unique constructions for inserting a laparoscopic/thorascopic grasper, dissector, or other laparoscopic/thorascopic surgical device into the body.

It is also an object of the invention to insert a laparoscopic/thorascopic surgical device into the body without the use of a separate trocar.

The objects of the invention can be achieved, as a whole or in part, by a laparoscopic tool for providing a passageway to a body cavity of a patient. The tool comprises an operating instrument for engaging a body part and an insertion device that punctures a covering of the body cavity to provide a passageway for access of the operating instrument to the body part. The insertion device includes a central passage extending inwardly from a proximal end thereof for frictionally receiving a shaft of the operating instrument. The insertion device also includes a distal end terminating in a sharp point for insertion of the insertion device and the operating instrument into the body cavity.

In a preferred embodiment of the invention, the insertion device is removed from the body cavity via another passageway to reduce chances of injury to internal body parts of the patient. The distal end of the insertion device may include a gripping region for engagement with a removal tool that removes the insertion device from the body cavity.

The objects of the invention can also be achieved, as a whole or in part, by a method for providing a passageway to access a body part within a body cavity of a patient. The method includes: connecting a central passage of an insertion device with a shaft of an operating instrument, puncturing a covering of the body cavity to form a first opening in the covering with a sharp end of the insertion device, inserting the entire insertion device and a portion of the shaft of the operating instrument through the covering and into the body cavity, separating the insertion device from the operating instrument within the body cavity, and removing the separated insertion device from the body cavity. The insertion device may be separated from the operating instrument by inserting a removal tool into the body cavity via a second opening, grasping the insertion device with the removal tool, and pulling the insertion device off of the operating instrument for removal from the operating area via the second opening.

The objects of the invention can also be achieved, as a whole or in part, by an insertion device for insertion into a body cavity of a patient. The insertion device includes a cylindrical shaft having a central passage extending inwardly from a proximal end for frictionally receiving an operating instrument, and a distal end having a conical shape terminating at a sharp point that punctures and forms a passageway through a covering of the body cavity to provide access for the operating instrument to a body part within the body cavity. The distal end includes a gripping region for engagement with a removal tool which removes the insertion device from the operation instrument within the body cavity. The gripping region can include a channel extending through the distal end adjacent the sharp point.

In another preferred embodiment, a laparoscopic tool includes an operating instrument adapted to engage a body part within a body cavity having a protective layer. The instrument includes a shaft at a distal end thereof and further includes an insertion cap having a central passage extending inwardly from a proximal end for frictionally receiving and covering the distal end of the operating instrument. The insertion cap also includes a distal end terminating in a blunt end adapted for insertion through an opening in the protective layer and for protecting the operating instrument during insertion of the operating instrument into the body cavity. The blunt end is unable to pierce the protective layer under normal human force exerted in a surgical environment, such as during laparoscopic surgery.

In yet another example of the present invention, a method for using a laparoscopic tool to access a body part within a body cavity of a patient is disclosed. The method includes connecting a central passage of an insertion cap with a shaft or distal end of an operating instrument, the combination of the insertion cap and the operating instrument forming the laparoscopic tool, splitting a covering of the body cavity to form a first opening in the covering with a separate cutting instrument, inserting the insertion cap and a portion of the operating instrument through the covering and into the body cavity, separating the insertion cap from the operating instrument within the body cavity, and removing the separated insertion cap from the body cavity.

Further scope of applicability of the present invention will become apparent from the description given hereinafter. However, it should be understood that the detailed description and specific examples, while indicating preferred embodiments of the invention, are given by way of illustration only, since the invention will become apparent to those skilled in the art from this detailed description.

### BRIEF DESCRIPTION OF THE DRAWINGS

The invention will be described in conjunction with the following drawings in which like-referenced numerals designate like-elements, and wherein:

FIG. 1 is an exploded isometric view showing a laparoscopic/thorascopic tool in accordance with the preferred embodiment of the invention;

FIG. 2 is an isometric view illustrating the laparoscopic/thorascopic tool of FIG. 1 and a removal tool;

FIG. 3 is a sectional view of the laparoscopic/thorascopic instrument taken along line 3-3 of FIG. 2;

FIG. 4 is an isometric view of the laparoscopic/thorascopic tool and a removal tool extended into a body cavity;

FIG. 5 is an isometric view of the laparoscopic/thorascopic tool and removal tool of FIG. 4 after separation of an insertion device from an operating instrument;

FIG. 6 is a side view of a laparoscopic/thorascopic tool inserted into a body cavity in accordance another embodiment of the invention;

FIG. 7 is an enlarged sectional view illustrating the laparoscopic/thorascopic tool of FIG. 6 inserted into the body cavity;

FIG. 8 is a side view illustrating a laparoscopic/thorascopic tool in accordance with another embodiment of the invention;

FIG. 9 is an enlarged sectional view illustrating the laparoscopic/thorascopic tool of FIG. 8; and

FIG. 10 is a sectional view of a laparoscopic/thorascopic instrument in accordance with yet another embodiment of the invention.

### DETAILED DESCRIPTION OF THE INVENTION

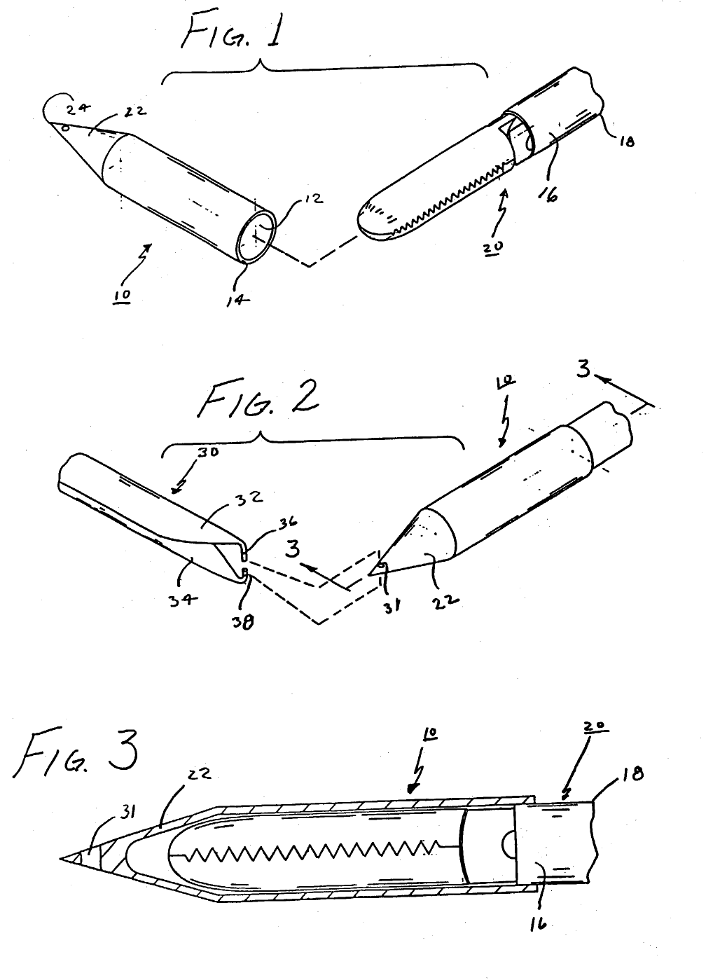

Referring to FIGS. 1 and 4, one preferred embodiment of the invention includes an insertion cap 10, in the general shape of a pointed tip or cone that can be similar in construction to a tip of the type employed on a target arrow. The insertion cap 10 has a central passage 12 extending inwardly from a proximal end 14 of the cap 10 for frictionally receiving an outer surface 16 of a shaft 18 of a laparoscopic device 20. A distal end 22 of the insertion cap 10 terminates at a sharp point 24 to permit the cap 10 to puncture a patient's skin 26 (FIG. 4), i.e., in the abdomen, to thereby insert the cap 10 and the laparoscopic device 20 attached thereto into the operating site 28.

After the laparoscopic device 20 with the cap 10 engaged thereon has been inserted into the operating site 28, it is important that the cap 10 be removed from the patient's body so that it does not injure internal body parts or organs. In the preferred embodiments of this invention, the cap or tip 10 is provided with a gripping area or region that can be engaged by a removal tool that initially is directed through a trocar inserted into the abdomen in another, adjacent area 40 of the patient's body.

In accordance with the broadest aspects of the invention, the cap 10 need not be provided with a separately definable gripping area or surface, and a variety of different instruments can be employed to pull the cap off of the device 20 and remove it from the patient's body. For example, a grasper known in the medical field as a Big Ass Grabber can be employed.

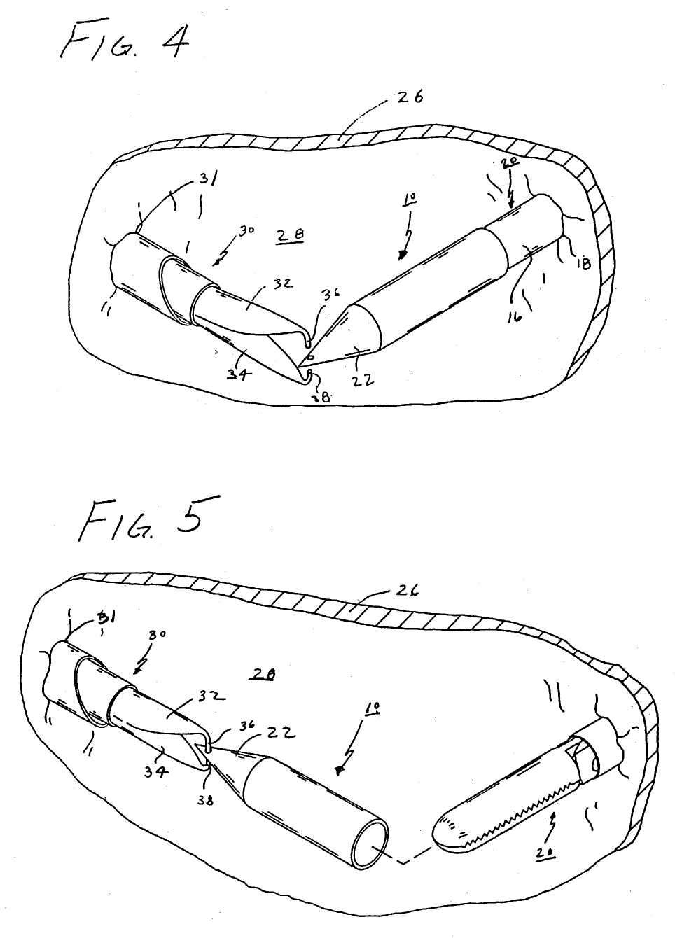

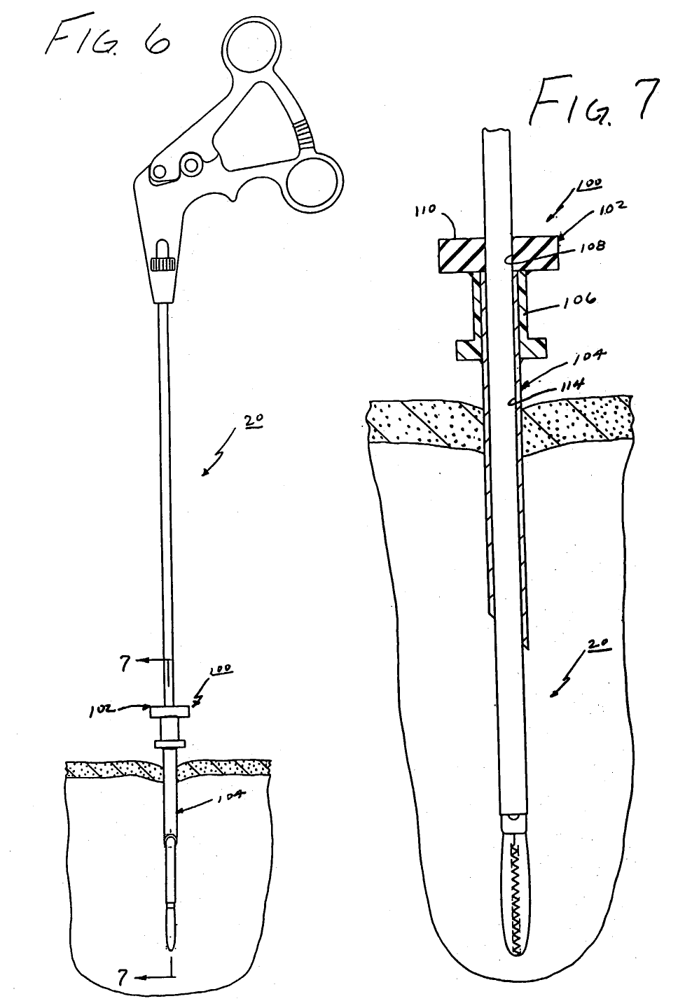

Referring to FIGS. 2-5, in the most preferred embodiment of the invention, the gripping area is a passage 31 through the distal end 22 of the cap 10 adjacent the point 24. A removal tool 30 includes opposed jaws 32, 34 having inturned legs 36, 38 that can be biased toward each other to extend through the passage 31 in the cap 10 from opposite sides thereof. Thereafter, as can be seen best in FIG. 5, the removal tool 30 is withdrawn to forceably pull the cap 10 off of the laparoscopic device 20 to which it previously was attached, and then remove the cap 10 from the patient's body.

In an alternative embodiment, which is believed to be less preferred than the above-disclosed embodiment because of the inclusion of parts that are movable relative to each other during the insertion operation, a needle insertion device is employed in place of the removable cap.

Specifically, referring to FIGS. 6 & 7, one embodiment of an insertion device is shown at 100. This insertion device 100 includes an upper cap section 102 made of a hard rubber or plastic material and a lower, hollow needle 104 having a width of, for example, about 14 gauge, and secured to the cap section 102. The needle 104 can be secured to the cap section 102 by having the needle's proximal end either friction fit into a downwardly extending, hollow hub 106 of the cap section 102, or threaded into a downwardly extending hollow threaded hub (not shown) of the cap section 102, as desired. The specific tool of securing the needle 104 to the cap section 102 is not considered to be a limitation on the broadest aspects of the present invention.

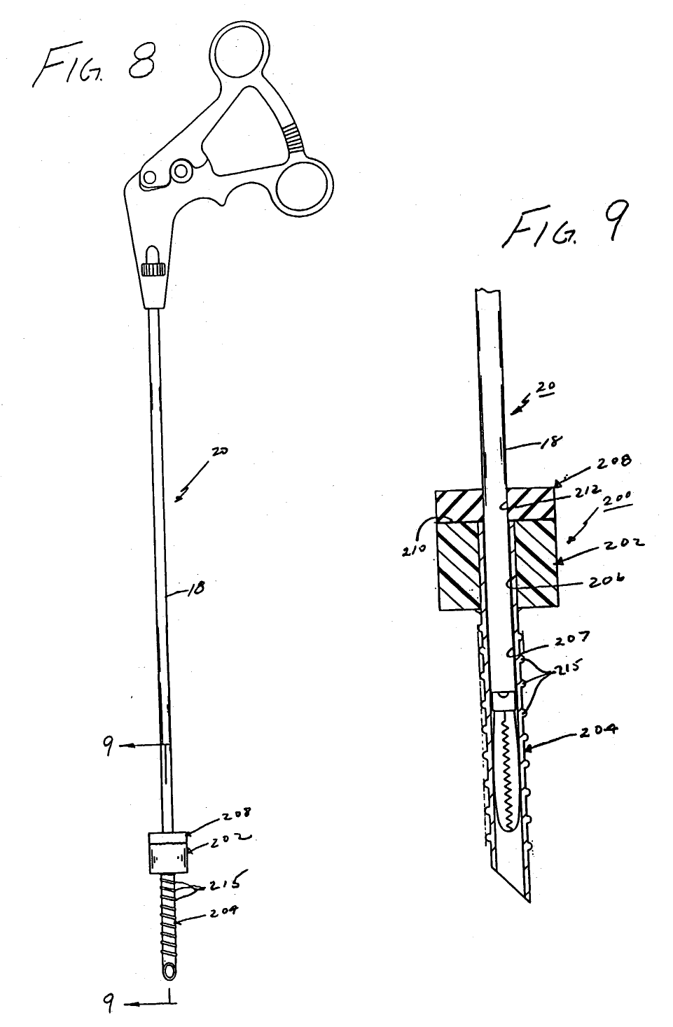

As can be seen best in FIG. 7, the cap section 102 is adhered to (by, for example, an adhesive or other desired attachment procedure) or integrally formed with a top surface of the hollow hub 106, and includes a central passageway 108 through an upper wall 110. This passageway 108 is aligned with, and is of a similar dimension to passageway 114 through the needle 104. The passageway 108 in the upper wall 110 of the cap section 102 and the passageway 114 through the needle 104 each has a diameter that is approximately the same as the outer diameter of a laparoscopic instrument 20 to be received within the insertion device 100. For example, when the shaft 18 of the laparoscopic instrument 20 has an outer diameter of approximately 5 mm., then the diameter of the passageway 108 through the upper wall 110 of the cap section 102 and through the aligned passageway 114 of the needle 104 of the insertion device 100 likewise will be approximately 5 mm.

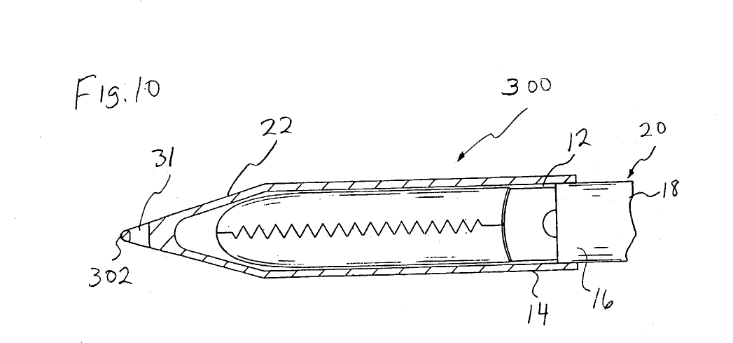

If desired, the outer surface of the needle 104 can be provided with threads to assist in both the insertion of the needle 104 through the patient's skin, and in retaining the needle 104 in a desired axial position in the patient. Such threads are shown at 215 in the embodiment of the invention illustrated in FIGS. 8 and 9, but are equally usable in the embodiment of FIGS. 6 and 7.

In use, the insertion device 100 can be inserted, by itself, through the patient's skin, and thereafter the laparoscopic instrument 20 inserted through the passageway (i.e., 108 and 114) of the insertion device into the surgical area of the body cavity. In this embodiment, the upper cap section 102 is sufficiently resilient to permit the shaft 18 of the laparoscopic instrument 20 to be passed through the insertion device 100, and also to provide a sufficient frictional grip on the shaft 18 of the laparoscopic instrument 20 to maintain the laparoscopic instrument 20 in a desired position for performing a surgical procedure.

Alternatively, the insertion device 100 can first be inserted on the shaft 18 of the laparoscopic instrument 20, and positioned on the shaft 18 so that the distal end of the needle 104 projects beyond the distal end of the laparoscopic instrument (e.g. see FIGS. 8 and 9). In this orientation, the insertion device 100 is employed to create a percutaneous puncture through the patient's skin, and thereafter, the laparoscopic instrument 20 is moved axially relative to the insertion device 100 to a desired location within the patient's body for providing a surgical procedure.

Referring to FIGS. 8 and 9, an alternative embodiment of an insertion device 100 is shown at 200. This insertion device 200 includes an upper, or proximal head section 202 and a downwardly directed, distal, hollow needle 204 formed (e.g., by molding) as a single unit. If desired, the needle 204 can be provided with threads 215 formed thereon to assist in both the insertion of the needle 204 through the patient's skin, and in retaining the needle 104 in a desired axial position in the patient. However, this is an optional feature of the invention.

A passage 206 extends through the head section 202 and an aligned passage 207 extends through the needle section 204 for receiving the laparoscopic instrument 20, in the same manner as described above in connection with the embodiment of FIGS. 6 and 7. A rubber, hollow, locator disk 208 is adhered (such as by an adhesive or any other desired attachment procedure) to the top surface 210 of the head section 202 of the insertion device 200, and includes a central passage 212 aligned with the passages 206 and 207 through the head section 202 and needle section 204, respectively. Alternatively, the locator disk 208 and head section 202 may be integrally formed of the same rubber or plastic material.

This locator disk 208 is designed to provide a sufficient frictional grip on the shaft 18 of the laparoscopic instrument 20 to maintain the laparoscopic instrument 20 in a desired orientation, or position, within a patient's body when a surgical procedure is being performed. In other words, the locator disk 208 provides the same function that is provided by the hard rubber or plastic upper cap section 102 of the insertion device 100.

It should be understood that the insertion devices 100 and 200, although shown as including linear needle sections 104 and 204, respectively, can include curved, hollow needle sections, as desired, to permit the reception of laparoscopic instruments having a similarly curved rigid shaft, or a shaft that is sufficiently flexible to conform to the curvature of the needle section.

In yet another alternative embodiment, a blunt ended insertion cap 300 is used instead of the sharp pointed insertion cap 10 described above. As shown by example in FIG. 10, the insertion cap 300 has the general shape of a rounded tip or cone that is otherwise similar in structure to the insertion cap 10 discussed above. The insertion cap 300 has a central passage 12 extending inwardly from a proximal end 14 of the cap 300 for frictionally receiving an outer surface 16 of a shaft 18 of the laparoscopic device 20. A distal end 22 of the insertion cap 300 terminates at a blunt or rounded tip 24 to permit the cap 300 to move within soft tissue of the patient and displace muscle fibers and blood vessels without lacerating the tissue, fibers or vessels, e.g., in the abdomen of the operating site 28.

The insertion cap 300 does not perforate the patient's skin as discussed above in relation to the cap 10. In use during surgery, an operator (e.g., surgeon) makes an incision through a patient's skin with a sharp tool, such as a scalpel. The cap 300 and attached laparoscopic device 20 can then be inserted through the incision into the patient's body. The cap 300 has it's tip 24 rounded to prevent it from being used as a piercing instrument. Accordingly, the cap 300 is not adapted to puncture and bore through the patient's skin as is the insertion cap 10 shown in FIGS. 1-5 and described above.

In areas of the body adjacent delicate organs, blood vessels, muscle fibers and other delicate body parts that would be threatened with injury if cut, it is preferable to use the cap 300 instead of sharp edged tools that perforate or lacerate the skin. To help prevent injury to delicate body parts from movement by a surgical tool, the insertion cap 300 is constructed so that it cannot pierce or puncture a patient's skin under human force or pressure in an operating environment.

As discussed above in reference to FIGS. 1-5, after the laparoscopic device 20 with the cap 10 engaged thereon has been inserted into the operating site 28, it is important that the cap 10 be removed from the patient's body so that it does not injure internal body parts or organs. However, as can be seen in FIG. 10, it is not as critical that the cap 300 be removed from the patient's body after insertion since the cap 300 has a rounded tip 24 that does not easily puncture delicate body parts. Yet the cap is foreign to the body, and preferably should be removed from the patient's body during a surgical procedure to reduce risk of infection to the patient or misplacement of the cap 300. Accordingly, in the preferred embodiments of this invention, the cap or tip 300 is provided with a gripping area or region that can be engaged by a removal tool that initially is directed through a trocar inserted into the abdomen in another, adjacent area 40 of the patient's body as discussed above for the other preferred embodiments.

In accordance with the broadest aspects of the invention, the cap 300 need not be provided with a separately definable gripping area or surface, and a variety of different instruments can be employed to pull the cap off of the device 20 and remove it from the patient's body. For example, a grasper known in the medical field as a Big Ass Grabber can be employed.

While not being limited to a particular theory, referring to FIG. 10, the preferred gripping area is a passage 31 through the distal end 22 of the cap 300 adjacent the tip 302. A removal tool 30 as shown in FIGS. 2, 4 and 5 includes opposed jaws 32, 34 having inturned legs 36, 38 that can be biased toward each other to extend through the passage 31 in the cap 300 from opposite sides thereof. Thereafter, similar to the arrangement shown in FIG. 5, the removal tool 30 could be withdrawn to forcibly pull the cap 300 off of the laparoscopic device 20 to which it previously was attached, and then remove the cap 300 from the patient's body.

It should be apparent from the aforementioned description and attached drawings that the concept of the present application may be readily applied to a variety of preferred embodiments, including those disclosed herein. Without further elaboration, the foregoing will also fully illustrate the invention that others may, by applying current or future knowledge, readily adapt the same for use under various conditions of service.

We claim:

1. A laparoscopic tool, comprising:
an operating instrument for engaging a body part within a body cavity having a protective layer, and including a shaft at a distal end thereof:
an insertion cap including a central passage extending inwardly from a proximal end for frictionally receiving and covering said distal end of said operating instrument, said insertion device also including a distal end terminating in a blunt end adapted for insertion through an opening in the protective layer and for protecting said operating instrument during insertion of said operating instrument into the body cavity, said blunt end being unable to pierce the protective layer under human force.

2. The laparoscopic tool of claim 1, further comprising a cutting tool for providing the opening in the protective layer.

3. The laparoscopic tool of claim 1, further comprising a trocar adapted for insertion through the covering of the body cavity to provide another passageway, and a removal tool adapted for insertion through said trocar to separate said insertion device from said operating instrument.

4. The laparoscopic tool of claim 3, wherein the insertion device is removed from the body cavity via the other passageway to reduce the chances of injury to internal body parts of the patient.

5. The laparoscopic tool of claim 3, wherein said distal end of said insertion device includes a gripping region for engagement with said removal tool for separation of said insertion device from said operating instrument and removal of said insertion device from the body cavity via the other passageway with the removal tool.

6. The laparoscopic tool of claim 5, wherein said gripping region includes a channel extending through said distal end of said insertion device adjacent the sharp end.

7. The laparoscopic of claim 6, wherein the removal tool includes an extending jaw having an interned leg to extend through the channel of said insertion device for removing the insertion device from the body cavity.

8. The laparoscopic of claim 6, wherein the removal tool includes opposed jaws having inturned legs biased towards each other to extend through said channel of said insertion device from opposite sides thereof to remove said insertion device from the body cavity.

9. A method for using a laparoscopic tool to access a body part within a body cavity of a patient, comprising
(a) connecting a central passage of an insertion cap with a shaft or distal end of an operating instrument, the combination of the insertion cap and the operating instrument forming the laparoscopic tool;
(b) splitting a covering of the body cavity to form a first opening in the covering with a separate cutting instrument;
(c) inserting the insertion cap and a portion of the operating instrument through the covering and into the body cavity;
(d) separating the insertion cap from the operating instrument within the body cavity; and
(e) removing the separated insertion cap from the body cavity.

10. The method of claim 9, wherein step (d) includes inserting a removal tool into the body cavity via a second opening, grasping the insertion cap with the removal tool, and pulling the insertion cap off of the operating instrument for removal of the insertion cap from the body cavity via the second opening.

11. A laparoscopic tool, comprising:
an operating instrument for engaging a body part within a body cavity, and including a shaft at a distal end thereof;
an insertion device adapted for providing a passageway to the body part, said insertion device including a central passage extending inwardly from a proximal end for frictionally receiving said distal end of said operating instrument, said insertion device also including a distal end terminating in a sharp end for punctuating a covering of the body cavity for insertion of the insertion device and the operating instrument into the body cavity;
a trocar adapted for insertion through the covering of the body cavity to provide another passageway; and
a removal tool adapted for insertion through said trocar to separate said insertion device from said operating instrument.

12. The laparoscopic tool of claim 11, wherein the insertion device is removed from the body cavity via the other passageway to reduce the chances of injury to internal body parts of the patient.

13. The laparoscopic tool of claim 12, wherein said distal end of said insertion device includes a gripping region for engagement with said removal tool for separation of said insertion device from said operating instrument and removal of said insertion device from the body cavity via the other passageway with the removal tool.

14. The laparoscopic tool of claim 13, wherein said gripping region includes a channel extending through said distal end of said insertion device adjacent the sharp end.

15. The laparoscopic of claim 14, wherein the removal tool includes an extending jaw having an interned leg to extend through the channel of said insertion device for removing the insertion device from the body cavity.

16. The laparoscopic of claim 14, wherein the removal tool includes opposed jaws having inturned legs biased towards each other to extend through said channel of said insertion device from opposite sides thereof to remove said insertion device from the body cavity.

17. The laparoscopic tool of claim 11, wherein said distal end of said insertion device has a conical shape extending from said central passage to said sharp end.

18. The laparoscopic tool of claim 11, wherein said sharp end of said distal end of said insertion device is closed.

19. The laparoscopic tool of claim 11, wherein said insertion device includes an elongated hollow needle having a cylindrical compartment extending from said central passage through said sharp end.

20. The laparoscopic tool of claim 19, wherein said insertion device further comprises an upper cap section defining said central passage and secured to said needle with said central passage aligned with and of similar dimension to said cylindrical compartment, said upper cap section having a diameter approximately equal to an outer diameter of said operating instrument at its distal end for frictional engagement of said insertion device with said operating instrument.

21. The laparoscopic tool of claim 20, wherein said upper cap section is sufficiently resilient to permit passage of said distal end of said operating instrument therethrough and to provide a sufficient frictional grip on said distal end of said operating instrument to maintain said operating instrument in a desired position for performing a surgical procedure.

22. The laparoscopic tool of claim 20, wherein said upper cap section includes a downwardly extending hollow hub frictionally fit about a proximal end of the needle.

23. The laparoscopic tool of claim 20, wherein said upper cap section includes a downwardly extending hollow hub having threads extending along an interior wall thereof, and said needle includes threads extending along an exterior wall thereof for threaded engagement with said interior wall of said hollow hub to secure said needle to said upper cap section.

24. The laparoscopic tool of claim 20, wherein said needle includes threads extending along an external wall thereof for rotatable engagement of said needle through the covering of the body cavity and securement of said needle in a desired axial position relative to the covering.
